(19) United States
(12) Patent Application Publication
(10) Pub. No.: US 2011/0226265 A1
(43) Pub. Date: Sep. 22, 2011

(54) CREATION AND IMPLANTATION OF AN ARTIFICIAL NAIL FOR THE TREATMENT OF DEFORMED OR MISSING NAILS

(76) Inventors: Mark Bauman, Marlton, NJ (US); James Nolan, Branford, CT (US)

(21) Appl. No.: 13/049,961

(22) Filed: Mar. 17, 2011

Related U.S. Application Data
(60) Provisional application No. 61/315,182, filed on Mar. 18, 2010.

(51) Int. Cl. A61B 17/00

(52) U.S. Cl. 128/898

(57) ABSTRACT

The creation and implantation of an artificial nail in the treatment of deformed or missing nails includes preparing the nail bed. A polypropylene mesh is applied and anchored to the nail bed. KeryFlex is applied to the polypropylene mesh.

### CROSS-REFERENCE TO RELATED APPLICATION

This application is based upon provisional application Ser. No. 61/315,182 filed Mar. 18, 2010, all of the details of which are incorporated herein by reference thereto.

### BACKGROUND OF INVENTION

The present invention relates to the creation of artificial nails. Various techniques have been used in the past to create artificial nails. These techniques have had differing degrees of effectiveness.

### SUMMARY OF THE INVENTION

An object of this invention is to provide techniques for the creation and implantation of an artificial nail for the treatment of deformed or missing nails.

### THE DRAWINGS

FIGS. 1-2 illustrate steps in the preparation of a nail bed in accordance with this invention;

FIGS. 3-6 illustrate artificial nails;

FIGS. 7-9 illustrate techniques for implanting an artificial nail; and

FIGS. 10-14 illustrate the steps involved in the creation and implantation of an artificial nail in accordance with this invention.

### DETAILED DESCRIPTION

The present invention is directed to the creation of artificial nails. It is to be understood that the practice of this invention may be accomplished for either toenails or fingernails.

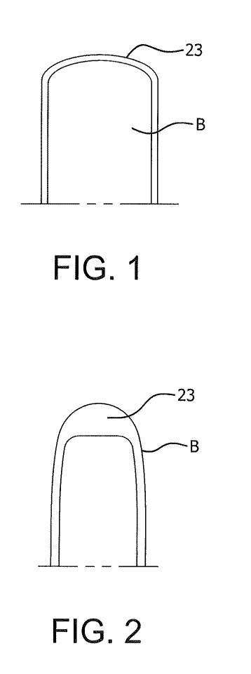

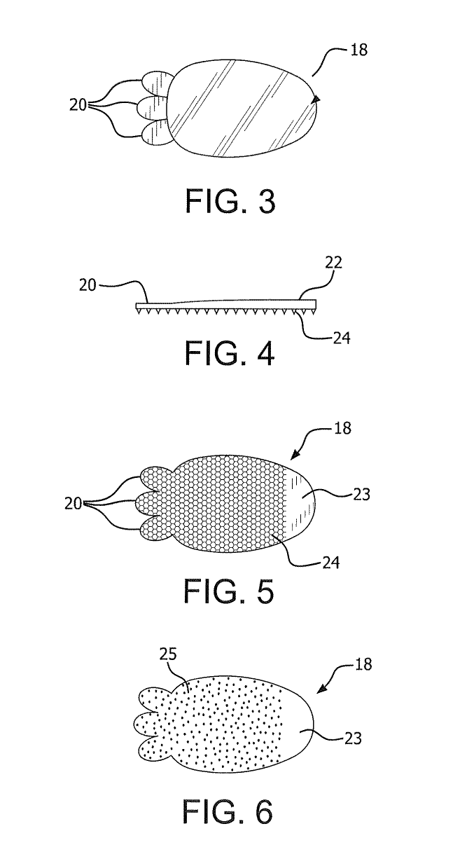

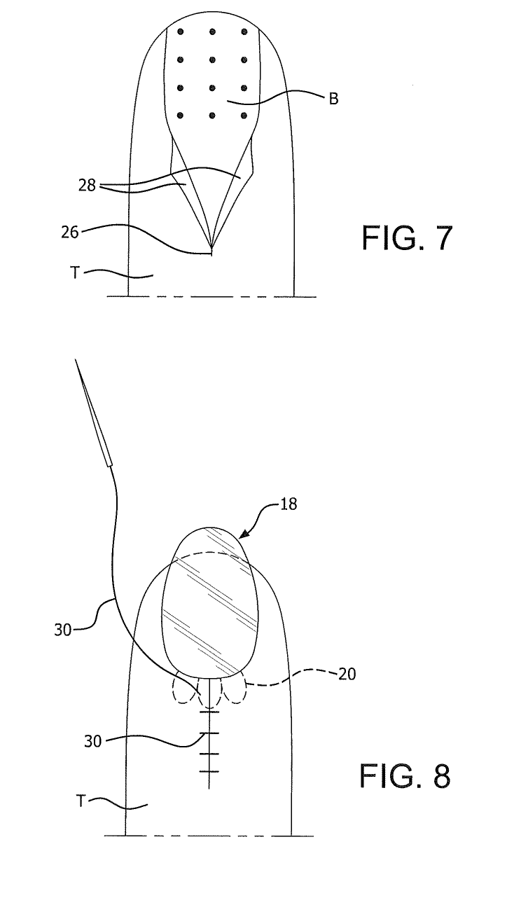

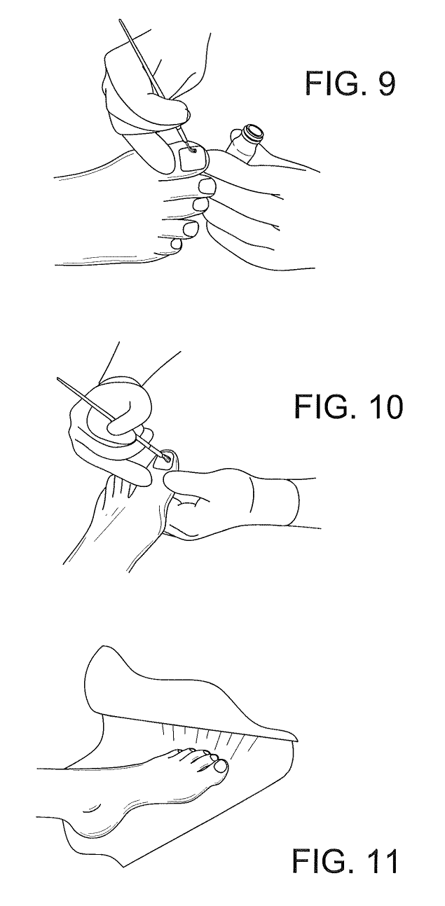

In general, the invention may be practiced by utilizing an established technique nail matricectomy which is performed by a chosen method on the patient's toe or finger while an appropriate tourniquet has already been placed around the digit, and the digit has been anesthetized . While the nail bed and matrix are excised sharply (See FIG. 1), leaving the most distal aspect of nail bed intact, the medial and lateral nail folds and eponychium are also incised sharply to create a U-shaped channel, with the open end of the “U” facing distally. See FIG. 2.

An appropriate-sized sterilized polypropylene mesh is selected. The mesh is carefully placed onto the digit and the medial and lateral wings are carefully placed under the medial and lateral nail folds, while the proximal anchor points are carefully placed under the exposed eponychium.

The eponychial area may be sutured at the discretion of the surgeon. At the level of the hyponychium, a continuous suture of a small gauge is placed between the mesh and most distal nail bed to create a distal anchorage of the mesh.

A non-adherent bandage is placed over the wound, and a compression bandage is placed over this bandage, while the tourniquet is released. The post-operative course is examined by the surgeon during the subsequent two weeks.

The artificial nail (18) works by allowing tissue ingrowth to occur. When the individual's digital nail is removed, collagen bonds are broken, causing bleeding and a wound. The healing mechanism for all wounds causes various chemicals to be released in an attempt to heal the wound. Ultimately a scab is formed. By placing the polypropylene mesh about the nail folds and eponychium, as well as at the hyponychium, a scaffolding mechanism will take place within the healing wound, “locking” the mesh to the surrounding tissues.

In instances where there is no existing nail, the cornified nail bed is removed (again leaving the most distal nail bed intact), causing the same type of wound and healing mechanism to occur.

The mesh is of optimal pore size to allow for ingrowth. Polypropylene is a preferred mesh material because it also has strong tear resistance, as well as being minimally reactive, so that little to no foreign body reaction should take place, eliminating the likelihood of rejection of the mesh.

When the matrix (i.e. the center growth or tissue of the nail) is either destroyed (via chemical, laser, etc.) or excised (such as in the preparation step of FIG. 1), the ability of the nail to continue to grow or regenerate is severely (if not completely) compromised. Therefore, with perfect matrix removal or destruction, no new nail forms, and the tissue which used to be nail, and which would have regenerated nail tissue, becomes cornified or as thicker skin tissue. This tissue becomes the platform on which KeryFlex can be applied, with the polypropylene mesh, with optimal porosity, acting as a perfect interface. Polypropylene mesh is applied and anchored to the nail bed (by preparing a large channel in the medial and lateral nail folds and eponychium, and in the hyponychium, where it is sutured with simple interrupted sutures)

KeryFlex is a nail restoration system, somewhat similar to a dental bond, whereby a bonding solution is applied (in this case) to the mesh, then the gel component is applied to the mesh, then the gel is cured via ultraviolet light, then more gel might be applied, then cured, then the sealant is applied, and then cured via ultraviolet light. This cured “nail” may then be gently shaped via emory board or drill. KeryFlex is currently used to fill in in poorly shaped or constituted nails by first applying the bonding solution to the existing nail, and then following the subsequent steps. KeryFlex cannot bond to a non-active or cornified nail bed; therefore, the mesh is implanted to act as a scaffold to allow KeryFlex to bond- or actually surround the mesh.

The procedure involves removal or destruction of the nail and nail matrix; when that is accomplished, the polypropylene mesh is implanted; when wound is stable (no drainage), KeryFlex is applied to the mesh that has been implanted to the digit.

At approximately two weeks, by which time the wound should have fully healed, the KeryFlex treatment should be ready for implementation. The nail bed will be prepared with an application of the bonding agent and allowed to dry for two minutes. See FIG. 10.

When the surface is dry, the chosen shade (clear, natural, or opaque) of resin material is carefully applied to the meshwork on the nail bed. This is gently sculpted to create a layer of “nail”. Care is taken to not allow the resin to fall onto tissue where there is no mesh. See FIG. 11.

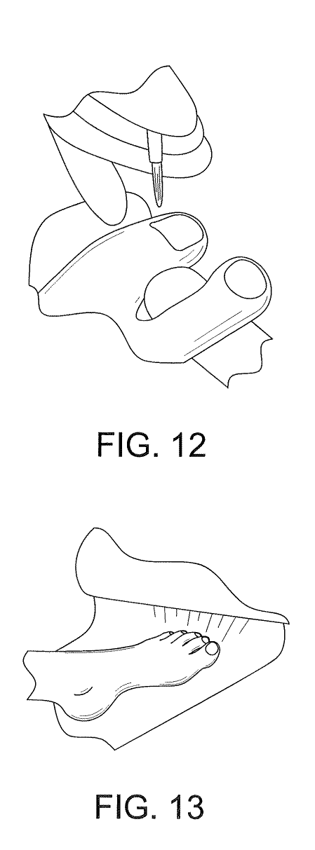

When the mesh is covered with resin to a reasonable depth and breadth, the resin is exposed to light from ultraviolet lamp for two minutes, causing the resin to polymerize and harden. See FIG. 12.

The practitioner determines if a second layer of resin is needed to create a thicker or fuller nail. If so, the same protocol is utilized as was done in the previous step, again taking care to not allow the resin to fall onto non-mesh tissue, and utilizing the ultraviolet lamp. The nail can then be “formed”, as necessary, using either a soft file or drill bur.

If the surgeon judges the new nail to be sufficient, corresponding to an acceptable level relative to the end of the toe, the sealing agent is then applied (see FIG. 13) and cured for two minutes with ultraviolet lamp. See FIG. 14.

Upon completion of this step, additional fine remodeling can be performed. Nail polish, at the discretion of the patient, can be applied almost immediately.

The materials of which the artificial nail is comprised are all FDA approved and have been used in the United States and other countries for some time. Medical grade polypropylene is used in, among other areas, hernia repair, vascular surgery, etc.

Only a known allergy to polypropylene, the chemicals which comprise KeryFlex, an open digital growth center, or any condition which would contraindicate the permanent removal of a digital nail and/or matrix would otherwise disqualify one's ability to undergo this procedure and treatment.

In the kit is the actual replacement polypropylene mesh. See FIG, 3.

FIG. 5 illustrates an artificial nail which is constructed as a layer of the resin material on the top of the collagen mesh on the underside as a cohering layer 24. See FIG. 4 for side view. This collagen mesh 24 extends distally to approximately 0.5 cm to the free edge 23 of the nail (and can be made longer, if desired, for toenails and custom shaped and sized fingernails).

As the nail 18 is attached to the nail bed B by cohesion, the bond is permanent and thus is stronger and more mechanically sound than simple adhesion. The three anchor points 20 on the proximal edge take up most of the mechanical stress which mimics a natural nail. The bonding which occurs peripherally is similar to what is found naturally that being the microadhesions which secure the nail plate to the underlying nail bed. Fibroblasts are generated from the created wound and should migrate along the collagen fibers which are adhered to the roughened, mesh-configured surface porous implant. Resin is chosen as the artificial nail material due to its inherent strength, history of biocompatibility, its ability to withstand erosion and abrasion, and its ability to be colored either industrially (to create the original color) or by items such as nail polish.

The procedure itself has no untoward effects, save the possible sensitivity one may have to the material (but the lack of a nail often creates a greater sensitivity which is quite evident post-operatively with patients who learn to accept it). If the nail 18 would need to be removed, an additional surgical intervention would be required. In the case of injury to the toe where the nail implant was partially destroyed or damaged, a replacement nail could be reapplied in the same fashion as the original implant surgery. The procedure affords the patient cosmetic relief for a perceived unsightly condition and enhances both beauty and self-assuredness and a general feeling of comfort in society. The invention provides a more permanent and acceptable alternative to the current status quo; it also increases by its very design and application, a higher level of acceptance on the part of the general population and affords a cosmetic as well as functional (as relates to sensation) correction of those who desire.

What is claimed is:

1. A method of creating and implanting an artificial nail in the treatment of deformed or missing nails comprising preparing a nail bed, applying a polypropylene mesh and applying KeryFlex to the mesh.
